(19) United States
(12) Patent Application Publication
(10) Pub. No.: US 2010/0189671 A1
(43) Pub. Date: Jul. 29, 2010

(54) INDOLE AMIDES AS PERFUMING INGREDIENTS

(75) Inventor: Jean-Marc Gaudin, Annemasse (FR)

Correspondence Address: WINSTON & STRAWN LLP;PATENT DEPARTMENT, 1700 K STREET, N.W., WASHINGTON, DC, 20006, US

(73) Assignee: Firmenich SA

(21) Appl. No.: 12/667,114

(22) PCT Filed: Jul. 29, 2008
(86) PCT No.: PCT/IB08/53030

§ 371 Date: Dec. 29, 2009

(30) Foreign Application Priority Data
Jul. 31, 2007 (IB) PCT/IB2007/053012

(51) Int. Cl. A61K 8/49; C07D 209/08; A61Q 5/02; A61Q 15/00; A61Q 13/00; C11D 3/50

(52) U.S. Cl. 424/65; 548/510; 424/70.1; 514/788; 512/10; 510/103; 512/4

(57) ABSTRACT

The present invention concerns the use of some indole amide derivatives as perfuming ingredients.

### TECHNICAL FIELD

The present invention relates to the field of perfumery. More particularly, it concerns the use of some indole amide derivatives as perfuming ingredients. The present invention concerns the use of said compounds in the perfumery industry as well as the compositions or articles containing said compounds.

### PRIOR ART

To the best of our knowledge, none of the present invention's compounds, or of their analogues, is known for a use as perfuming ingredient.

1-Acetyl-1H-indole is known from the prior art (e.g. Depree G. J. et al. in Journal of Organometallic Chemistry (2006), 691(4), 667-679) and is reported as simple chemical. No mention or suggestion is provided for its use in perfumery.

1-Propionyl-1H-indole is also known from the prior art (e.g. Ito Y. et al. in Bulletin of the Chemical Society of Japan (1984), 57(1), 73-84, Table 6, compound 30a-V). This compound is also reported as simple chemical and no mention or suggestion is provided for its use in perfumery.

### DESCRIPTION OF THE INVENTION

We have now surprisingly discovered that a compound of formula

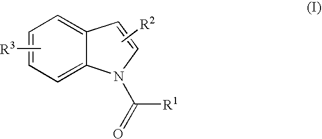

wherein R¹ represents a C₁-C₄ alkyl group; and
R³, which is a substituent on the 6 member ring, represents a hydrogen atom or a C₁-C₄ alkyl group, and R², which is a substituent on the 5 member ring, represents a hydrogen atom; or
R² represents a hydrogen atom or a C₁-C₆ alkyl group, and R³ represents a hydrogen atom;
   can be used as perfuming ingredient, for instance to impart various odor notes.

According to a particular embodiment of the invention, the compounds of formula (I) can be those wherein R¹ represents a C₁-C₄ alkyl group and both R² and R³ represent a hydrogen atom each.

As typical example of the invention, one may cite 1-acetyl-1H-indole which possesses an odor of the floral type. In particular it imparts a unique marriage of jasmine note and orange flower note, reminding a white flower bouquet providing to the perfuming compositions a natural touch. The invention's compound has also an interesting balsamic note which is capable of providing warmth and volume to the perfuming compositions to which it is added.

The odor of this compound, when compared to the one of indole, is much more complex since the latter is able to impart essentially jasmine type notes only and does not possess balsamic notes.

As another example of the compounds of the invention, one may cite 1-propionyl-1H-indole, which also possesses an odor similar to 1-acetyl-1H-indole, with jasmine and orange flower notes, with an additional naphthalene, woody note.

The odor of this compound is different from the one of indole in that the latter lacks the orange flower and woody notes.

As a further example of the invention, one may cite 1-butyryl-1H-indole, which possesses a floral odor with jasmine and hyacinth notes.

The odor of this compound is also more complex than the odor of indole, which lacks the hyacinth note.

As mentioned above, the invention concerns the use of a compound of formula (I) as perfuming ingredient. In other words it concerns a method to confer, enhance, improve or modify the odor properties of a perfuming composition or of a perfumed article, which method comprises adding to said composition or article an effective amount of at least a compound of formula (I). By “use of a compound of formula (I)” it has to be understood here also the use of any composition containing compound (I) and which can be advantageously employed in perfumery industry as active ingredients.

Said compositions, which in fact can be advantageously employed as perfuming ingredient, are also an object of the present invention.

Therefore, another object of the present invention is a perfuming composition comprising:
i) as perfuming ingredient, at least one invention's compound as defined above;
ii) at least one ingredient selected from the group consisting of a perfumery carrier and a perfumery base; and
iii) optionally at least one perfumery adjuvant.

By “perfumery carrier” we mean here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting example solvents such as dipropyleneglycol, diethyl phthalate, isopropyl myristate, benzyl benzoate, 2-(2-ethoxyethoxy)-1-ethanol or ethyl citrate, which are the most commonly used.

As solid carrier one may cite, as non-limiting examples, absorbing gums or polymers, or yet encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloids: Stabilisatoren, Dickungs- und Gehermittel in Lebensmittel, Band 2 der Schriftenreihe Lebensmittelchemie, Lebensmittelqualität, Behr's VerlagGmbH & Co., Hamburg, 1996. The encapsulation is a well known process to a person skilled in the art, and may be performed, for instance, using techniques such as spray-drying, agglomeration or yet extrusion; or consists of a coating encapsulation, including coacervation and complex coacervation techniques.

By “perfumery base” we mean here a composition comprising at least one perfuming co-ingredient.

Said perfuming co-ingredient is not of the formula (I). Moreover, by “perfuming co-ingredient” it is meant here a compound, which is used in perfuming preparation or composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of its general knowledge and according to intended use or application and the desired organoleptic effect. In general terms, these perfuming co-ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

For the compositions which comprise both a perfumery carrier and a perfumery base, other suitable perfumery carrier, than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company).

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

An invention's composition consisting of at least one compound of formula (I) and at least one perfumery carrier represents a particular embodiment of the invention as well as a perfuming composition comprising at least one compound of formula (I), at least one perfumery carrier, at least one perfumery base, and optionally at least one perfumery adjuvant.

It is useful to mention here that the possibility to have, in the compositions mentioned above, more than one compound of formula (I) is important as it enables the perfumer to prepare accords, perfumes, possessing the odor tonality of various compounds of the invention, creating thus new tools for their work.

Preferably, any mixture resulting directly from a chemical synthesis, e.g. without an adequate purification, in which the compound of the invention would be involved as a starting, intermediate or end-product, could not be considered as a perfuming composition according to the invention.

Furthermore, the invention's compound can also be advantageously used in all the fields of modern perfumery to positively impart or modify the odor of a consumer product into which said compound (I) is added. Consequently, a perfumed article comprising:
i) as perfuming ingredient, at least one compound of formula (I), as defined above, or an invention's perfuming composition; and
ii) a consumer product base;
   is also an object of the present invention.

For the sake of clarity, it has to be mentioned that, by “consumer product base” we mean here a consumer product, which is compatible with perfuming ingredients. In other words, a perfumed article according to the invention comprises the functional formulation, as well as optionally additional benefit agents, corresponding to a consumer product, e.g. a detergent or an air freshener, and an olfactive effective amount of at least one invention's compound.

The nature and type of the constituents of the consumer product do not warrant a more detailed description here, which in any case would not be exhaustive, the skilled person being able to select them on the basis of its general knowledge and according to the nature and the desired effect of said product.

Examples of suitable consumer product bases include solid or liquid detergents and fabric softeners as well as all the other articles common in perfumery, namely perfumes, colognes or after-shave lotions, perfumed soaps, shower or bath salts, mousses, oils or gels, hygiene products or hair care products such as shampoos, body-care products, deodorants or antiperspirants, air fresheners and also cosmetic preparations. As detergents there are intended applications such as detergent compositions or cleaning products for washing up or for cleaning various surfaces, e.g. intended for textile, dish or hard-surface treatment, whether they are intended for domestic or industrial use. Other perfumed articles are fabric refreshers, ironing waters, papers, wipes or bleaches.

Some of the above-mentioned consumer product bases may represent an aggressive medium for the invention's compound, so that it may be necessary to protect the latter from premature decomposition, for example by encapsulation.

The proportions in which the compounds according to the invention can be incorporated into the various aforementioned articles or compositions vary within a wide range of values. These values are dependent on the nature of the article to be perfumed and on the desired organoleptic effect as well as the nature of the co-ingredients in a given base when the compounds according to the invention are mixed with perfuming co-ingredients, solvents or additives commonly used in the art.

For example, in the case of perfuming compositions, typical concentrations are in the order of 0.01% to 10% by weight, or even more, of the compounds of the invention based on the weight of the composition into which they are incorporated. Concentrations lower than these, such as in the order of 0.001% to 5% by weight, can be used when these compounds are incorporated into perfumed articles, percentage being relative to the weight of the article.

The invention's compounds can be prepared according to a method comprising the step of acetylating, e.g. with a suitable acid chloride, the corresponding indole derivative.

The compounds 1-acetyl-1H-indole and 1-(1-oxopropyl)-1H-indole are known from the prior art.

### EXAMPLES

The invention will now be described in further detail by way of the following examples, wherein the abbreviations have the usual meaning in the art; the NMR spectral data were recorded in CDCl₃ with a 400 MHz machine for ¹H and ¹³C, the chemical displacement δ are indicated in ppm with respect to TMS as standard, the coupling constants J are expressed in Hz.

### Example 1

### Preparation of a Perfuming Composition

A perfuming composition for a detergent was prepared by admixing the following ingredients:

| Ingredient | Parts by weight |
|---|---|
| Geranyl acetate | 25 |
| Prenyl acetate | 5 |
| MNA aldehyde | 5 |
| Methyl anthranilate | 5 |
| 10%* Cetalox ®¹⁾ | 15 |
| Verdyl acetate | 150 |
| Verdyl proprionate | 25 |
| Coumarine | 25 |
| Dihydromyrcenol | 250 |
| Geraniol | 50 |
| Iso E Super ®²⁾ | 100 |
| Lorysia ®³⁾ | 150 |
| 10%* Neobutenone ®⁴⁾ alpha | 5 |
| Rose oxide | 5 |
| Verdox ®⁵⁾ | 150 |
| | 965 |

*in dipropyleneglycol
¹⁾8,12-epoxy-13,14,15,16-tetranorlabdane; origin: Firmenich SA, Geneva, Switzerland
²⁾1-(octahydro-2,3,8,8-tetramethyl-2-naphtalenyl)-1-ethanone; origin: International Flavors & Fragrances, USA
³⁾4-(1,1-dimethylethyl)-1-cyclohexyle acetate; origin: Firmenich SA, Geneva, Switzerland
⁴⁾1-(5,5-dimethyl-1-cyclohexen-1-yl)-4-penten-1-one; origin: Firmenich SA, Geneva, Switzerland
⁵⁾2-tert-butyl-1-cyclohexyl acetate; origin: International Flavors & Fragrances, USA

The addition of 35 parts by weight of 1-acetyl-1H-indole to the above-described perfuming composition reinforced considerably the strength, the warm and the natural side of the white flower aspect since the orange flower note became noticeable.
When to the above-mentioned composition was added the same amount of methylnaphtylketone, the fragrance was less “white flower bouquet”, less floral and more aggressive.

### Example 2

### Preparation of a Perfuming Composition

A perfuming composition of the jasmin type was prepared by admixing the following ingredients:

| Ingredient | Parts by weight |
|---|---|
| Benzyl acetate | 50 |
| Amyl cinnamic aldehyde | 10 |
| 1%* C 10 Aldehyde | 3 |
| 1%* Gamma undecalactone | 10 |
| 10%* Gamma nonalactone | 5 |
| Linalool | 10 |
| Phenethylol | 5 |
| Ylang I | 2 |
| | 995 |

*in dipropyleneglycol

The addition of 5 parts by weight of 1-acetyl-1H-indole (10% in dipropyleneglycol) to the above-described perfuming composition reinforced considerably the strength, the warm and the natural side of the white flower aspect since the orange flower note was much stronger.
When to the above-mentioned composition was added the same amount of indole (1% in dipropyleneglycol), the fragrance was less natural and warm due to the presence of a naphtalenic note of indole.

### Example 3

### Synthesis of 1-butyryl-1H-indole

The compound 1-butyryl-1H-indole has been prepared according to the method described for the synthesis of 1-Propionyl-1H-indole in Ito Y. et al. in Bulletin of the Chemical Society of Japan (1984), 57(1), 73-84, Table 6.

¹H-NMR: 1.05 (t, J=6, 3H); 1.82 (dq, J=6, J=6, 2H); 2.81 (d, J=6, 2H); 6.58 (d, J=2, 2H); 7.24 (dd, J=6, J=3, 1H); 7.33 (dd, J=6, J=3, 1H); 7.39 (d, J=2, 1H); 7.53 (d, J=6, 1H); 8.47 (d, J=6, 1H).

¹³C NMR: 13.8 (q); 18.1 (t); 37.6 (t); 108.9 (d); 116.6 (d); 120.8 (d); 123.5 (d); 124.6 (d); 125.0 (d); 130.3 (s); 135.6 (s); 171.4 (s).

EI-MS: 187 (19.4, M+.); 118 (10.2); 117 (100); 116 (6.9); 90 (8.6); 89 (11.4); 71 (2.5); 63 (4.2); 43 (8.1); 41 (3.5).

1.-5. (canceled)

6. A compound of formula 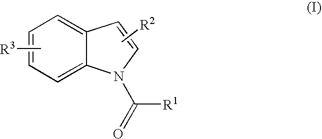
wherein R¹ represents a C₁-C₄ alkyl group; and
R³, which is a substituent on the 6 member ring, represents a hydrogen atom or a C₁-C₄ alkyl group, and R², which is a substituent on the 5 member ring, represents a hydrogen atom; or
R² represents a hydrogen atom or a C₁-C₆ alkyl group, and R³ represents a hydrogen atom;
provided that 1-acetyl-1H-indole and 1-propionyl-1H-indole are excluded.

7. A method to confer, enhance, improve or modify the odor properties of a perfuming composition or of a perfumed article, which method comprises adding to said composition or article an effective amount of at least a compound of formula (I) 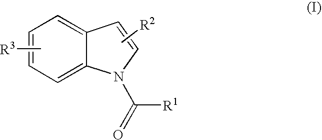
wherein R¹ represents a C₁-C₄ alkyl group; and
R³, which is a substituent on the 6 member ring, represents a hydrogen atom or a C₁-C₄ alkyl group, and R², which is a substituent on the 5 member ring, represents a hydrogen atom; or
R² represents a hydrogen atom or a C₁-C₆ alkyl group, and R³ represents a hydrogen atom.

8. A perfuming composition comprising
i) at least a compound of formula 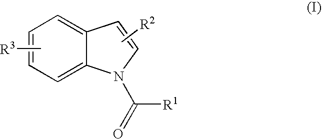
wherein R¹ represents a C₁-C₄ alkyl group; and
R³, which is a substituent on the 6 member ring, represents a hydrogen atom or a C₁-C₄ alkyl group, and R², which is a substituent on the 5 member ring, represents a hydrogen atom; or
R² represents a hydrogen atom or a C₁-C₆ alkyl group, and R³ represents a hydrogen atom;
ii) at least one ingredient selected from the group consisting of a perfumery carrier and a perfumery base; and
iii) optionally at least one perfumery adjuvant.

9. A perfumed article comprising:
i) at least a compound of formula 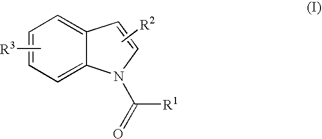
wherein R¹ represents a C₁-C₄ alkyl group; and
R³, which is a substituent on the 6 member ring, represents a hydrogen atom or a C₁-C₄ alkyl group, and R², which is a substituent on the 5 member ring, represents a hydrogen atom; or
R² represents a hydrogen atom or a C₁-C₆ alkyl group, and R³ represents a hydrogen atom; and
ii) a consumer product base.

10. The perfumed article according to claim 9, wherein the consumer product base is a solid or liquid detergent, a fabric softener, a perfume, a cologne or after-shave lotion, a perfumed soap, a shower or bath salt, mousse, oil or gel, a hygiene product, a hair care product, a shampoo, a body-care product, a deodorant or antiperspirant, an air freshener, a cosmetic preparation, a fabric refresher, an ironing water, a paper, a wipe or a bleach.
